(19) United States
(12) Patent Application Publication
(10) Pub. No.: US 2018/0282339 A1
(43) Pub. Date: Oct. 4, 2018

(54) PROCESS FOR THE PREPARATION OF THE AMORPHOUS FORM OF IBRUTINIB AND NOVEL CRYSTALLINE FORM

(71) Applicant: OLON S.P.A., Rodano MI (IT)

(72) Inventors: Giorgio BERTOLINI, Rodano MI (IT); Lazzaro FELICIANI, Rodano MI (IT); Ilaria FERRANDO, Lugano (CH); Mara SADA, Rodano MI (IT)

(21) Appl. No.: 15/772,140

(22) PCT Filed: Nov. 16, 2016
(86) PCT No.: PCT/IB2016/056881

§ 371 Date: Apr. 30, 2018

(30) Foreign Application Priority Data
Nov. 16, 2015 (IT) UB2015A005616

(51) Int. Cl. C07D 487/04

(52) U.S. Cl. CPC C07D 487/04; C07B 2200/13

(57) ABSTRACT

Subject-matter of the invention is a process for the preparation of the amorphous form of ibrutinib and a novel crystalline form.

### ABSTRACT OF THE INVENTION

Subject-matter of the invention is a process for the preparation of the amorphous form of ibrutinib and a novel crystalline form.

### BACKGROUND ART

Ibrutinib is an antitumor compound, currently used in the therapy of some lymphomas. Its International Nonproprietary Name (INN) is 1-[(3R)-3-[4-amino-3-(4-phenoxyphenyl)-1H-pyrazolo[3,4-d]pyrimidin-1-yl]piperidin-1-yl]prop-2-en-1-one and has the following structural formula:

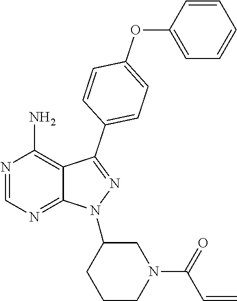 [SMILES: C=CC(=O)N1CCCC(n2nc(-c3ccc(Oc4ccccc4)cc3)c3c(N)ncnc32)C1]

Various crystalline forms of ibrutinib and its amorphous form have been described in WO2015/081180, WO2013/184572 and in “ip.com Number IPCOM000238881D”. In this document, the amorphous form was obtained by drying a solution of ibrutinib in acetone or methyl-tetrahydrofuran under an air flow, whereas in WO2013/184572 it is obtained by dissolving the Form A of ibrutinib in dichloromethane and quickly evaporating by means of rotary evaporator.

### OBJECTS OF THE INVENTION

An object of the invention is to provide novel processes for the preparation of the amorphous form of ibrutinib, which are reproducible and industrially convenient. Another object of the invention is to provide a novel crystalline form of ibrutinib and the processes for the preparation thereof.

### BRIEF DESCRIPTION OF THE FIGURES

FIG. 1 shows the XRPD spectrum of the amorphous form of ibrutinib.

FIG. 2 shows the FT-IR spectrum of the amorphous form of ibrutinib.

FIG. 3 shows the DSC profile of the amorphous form of ibrutinib.

FIG. 4 shows the XRPD spectrum of the novel Form L of ibrutinib.

FIG. 5 shows the FT-IR spectrum of the novel Form L of ibrutinib.

FIG. 6 shows the DSC profile of the novel Form L of ibrutinib.

### DESCRIPTION OF THE INVENTION

According to one of its aspects, subject-matter of the invention is a process for the preparation of the amorphous form of ibrutinib, comprising dissolving ibrutinib in a solvent selected from 1,2-dimethoxy-ethane and ethanol until obtaining a saturated solution, adding water to said solution and isolating the so-obtained precipitate.

The saturated solution can be obtained by dissolving ibrutinib in the solvent at room temperature.

Alternatively to the process described above, the amorphous form of ibrutinib can be obtained by evaporating an advantageously not-saturated solution of ibrutinib in one or more solvents, for example in a solvent selected from 1,4-dioxane, methyl ethyl ketone, methanol, dimethylsulfoxide, ethanol, 2-butanol, acetonitrile, ethyl acetate, nitromethane, 2-methoxyethanol, 1,2-dimethoxy-ethane, dimethylformamide, methylene chloride and acetone. 1,2-Dimethoxy-ethane can only be used in mixture with other solvents, as it will be seen below.

Solvents as 1,4-dioxane, methyl ethyl ketone are preferred and when one is working with said solvents, the evaporation of the solution can be substantially carried out at any temperature and pressure. By way of example, one can work in the following conditions:
low temperature and room pressure (4-10° C./1 atm)
room temperature and pressure (17-25° C./1 atm)
high temperature and room pressure (60° C./1 atm)
room temperature and low pressure (17-25° C./10−2 atm)
high temperature and low pressure (40° C./10−2 atm)

On the contrary, when one workswith the other solvents mentioned above, the evaporation is carried out in the following conditions:
temperature and pressure 17-25° C./1 atm in methanol, acetone;
temperature and pressure 60° C./1 atm in a solvent selected from 2-butanol, 2-methoxyethanol, acetonitrile, dimethylformamide, dimethylsulfoxide, and ethyl acetate;
temperature and pressure 17-25° C./10⁻² atm in a solvent selected from 2-butanol, acetonitrile, methylene chloride, methanol, ethanol, nitromethane and ethyl acetate;
temperature and pressure 40° C./10⁻² atm in a solvent selected from 2-butanol, 2-methoxyethanol, acetonitrile, dimethylformamide, dimethylsulfoxide, ethyl acetate, ethanol and nitromethane;

As mentioned, it is also possible to evaporate a solvent mixture for obtaining the amorphous form. Preferred mixtures of solvents are the following:
methyl ethyl ketone/1,2-dimethoxy-ethane;
methyl ethyl ketone/1,4-dioxane;

It is also possible to obtain the amorphous form by evaporation at room temperature, at a temperature around 60° C. and room pressure, or at low pressure (about 10⁻² atmospheres) at room temperature or about 40° C., in the following mixtures of solvents:
2-propanol/1,4-dioxane;
methyl ethyl ketone/2-propanol;
methyl ethyl ketone/acetonitrile; and
methyl ethyl ketone/ethanol.

It is also possible to obtain the amorphous form by evaporation at a temperature around 60° C. and room pressure, or at low pressure (about 10⁻² atmospheres) at room temperature or about 40° C., in the following mixtures of solvents:
methyl ethyl ketone/ethyl acetate;
methyl ethyl ketone/2-butanol;
2-propanol/1,2-dimethoxy-ethane;
2-propanol/ethyl acetate; and
2-propanol/2-butanol.

It is also possible to obtain the amorphous form by evaporation at room pressure and temperature or at about 40° C. at low pressure (about 10⁻² atmospheres), in the following solvent mixture:
2-propanol/acetonitrile

It is also possible to obtain the amorphous form by evaporation at 60° C. and room pressure or at about 40° C. at low pressure (about 10⁻² atmospheres), in the following solvent mixture
2-propanol/ethanol

It is also possible to obtain the amorphous form by evaporation at room pressure and temperature or at room temperature and low pressure (about 10⁻² atmospheres) or at low temperature (4-10° C.), in the following solvent mixture:
methyl ethyl ketone/acetone.

Finally, it is also possible to obtain the amorphous form by evaporation at room pressure and temperature or at room temperature and low pressure (about 10⁻² atmospheres), in the following solvent mixture:
2-propanol/acetone.

The amorphous form of ibrutinib obtainable and/or obtained by the processes described above is a further subject-matter of the invention.

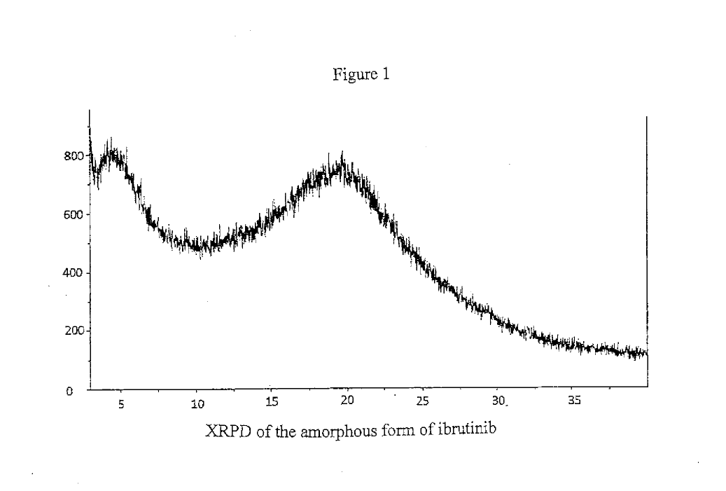

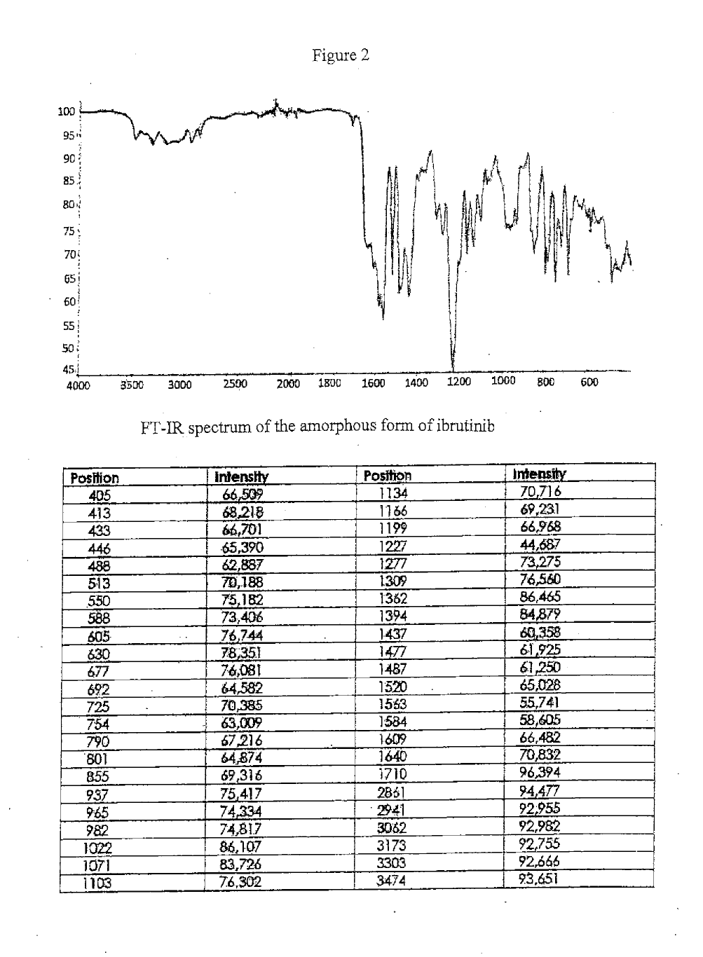

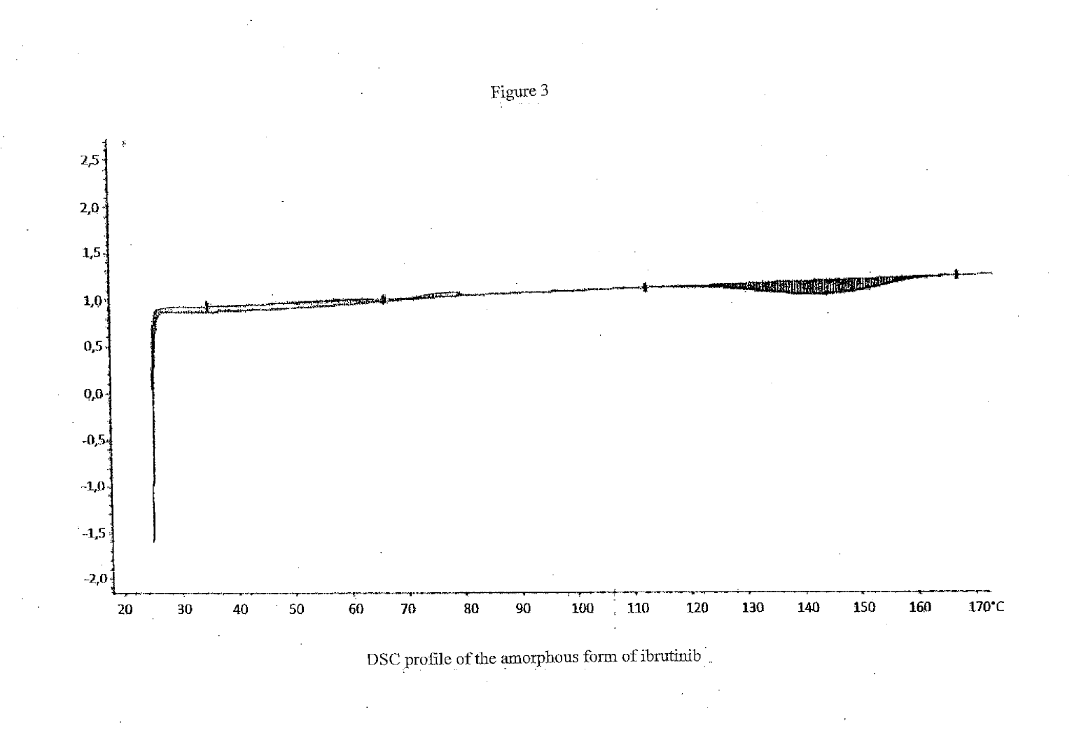

The XRPD spectrum of the amorphous form is shown in FIG. 1 and shows that there is no crystalline form; the FT-IR spectrum is depicted in FIG. 2 and the DSC profile is depicted in FIG. 3. The amorphous form of ibrutinib, characterized by said FT-IR spectrum and said DSC profile, is a further subject-matter of the invention.

As it can be appreciated by the DSC analysis, the amorphous form obtained by the process described above shows an endothermic peak at about 58° C. due to trapped water. During a second heating, an exothermic peak is detected at about 144° C. due to the degradation of the molecule.

The amorphous form is particularly stable, both to grinding and kneading, and to exposure to various combinations of temperature and humidity.

Subject-matter of the invention, according to another aspect thereof, is the use of the amorphous form of ibrutinib in therapy and particularly in the treatment of tumors as lymphomas and leukemias.

Subject-matter of the invention is also a pharmaceutical composition comprising the amorphous form of ibrutinib together with conventional carriers and/or excipients, preferably an oral composition, for example a tablet or a capsule. Such compositions will comprise 40 to 300 mg amorphous form of ibrutinib, for example 120-150 mg, advantageously about 140 mg and will be administered 1 to 5 times per day, advantageously 3 times per day. However, other dosages and administration could be provided, depending on pathology and conditions of the subject to be treated.

Subject-matter of the invention, according to another aspect thereof, is a method for treating tumors, as lymphomas and leukemias, comprising administering an effective dose of the amorphous form of ibrutinib to a subject in the need thereof.

By “subject” herein is meant a mammal, preferably a human.

Subject-matter of the invention, according to another aspect thereof, is a solvate of ibrutinib with 1,2-dimethoxy-ethane.

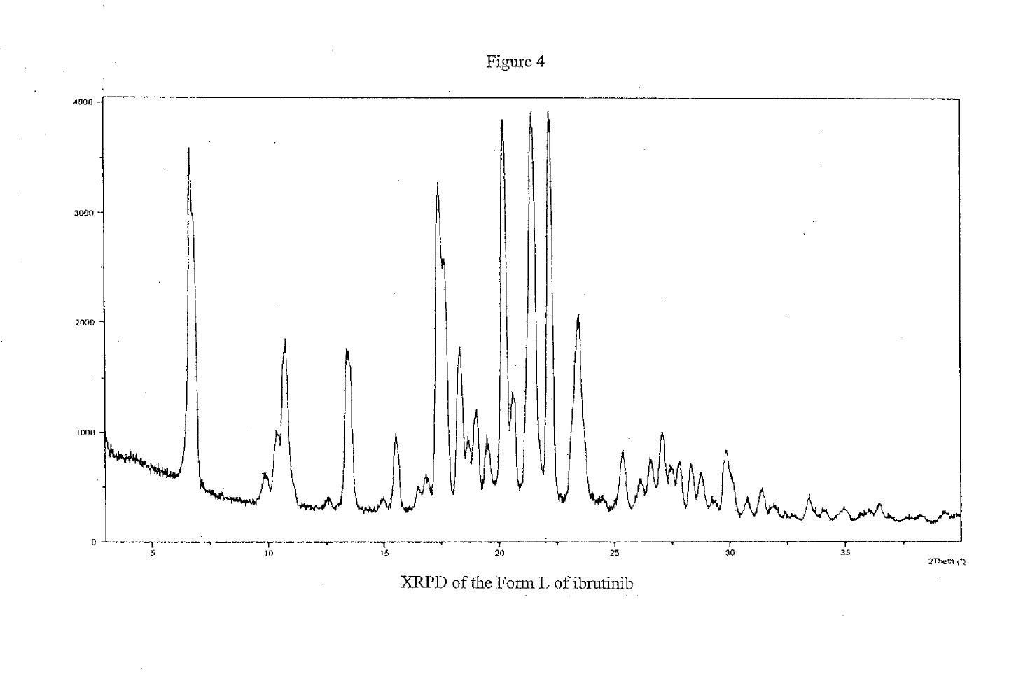

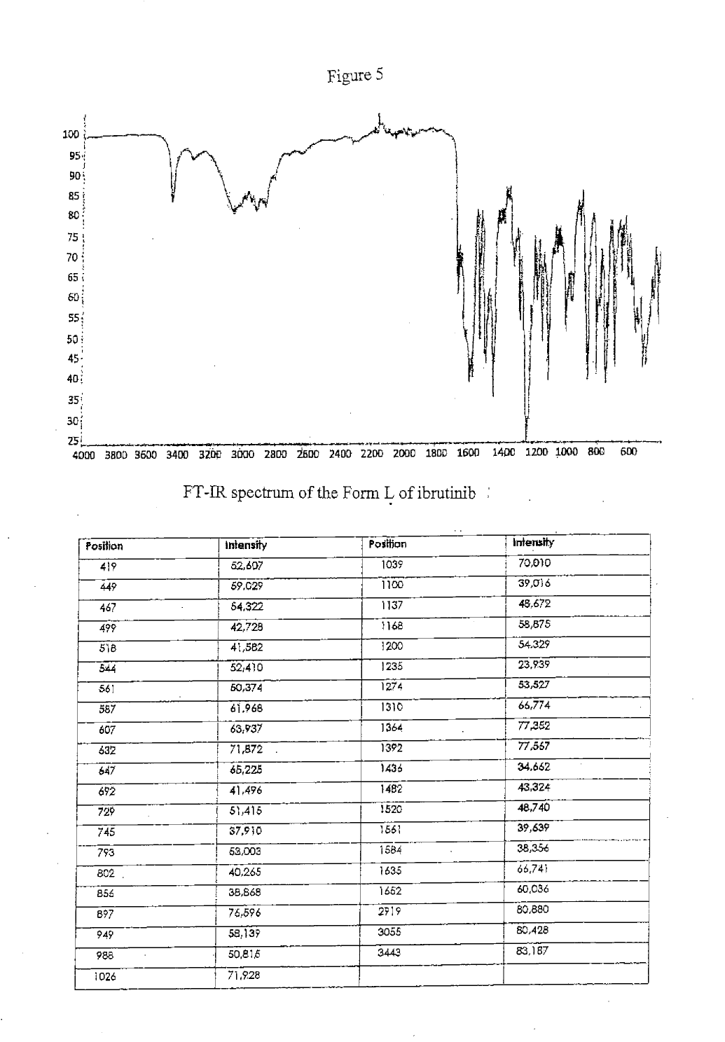

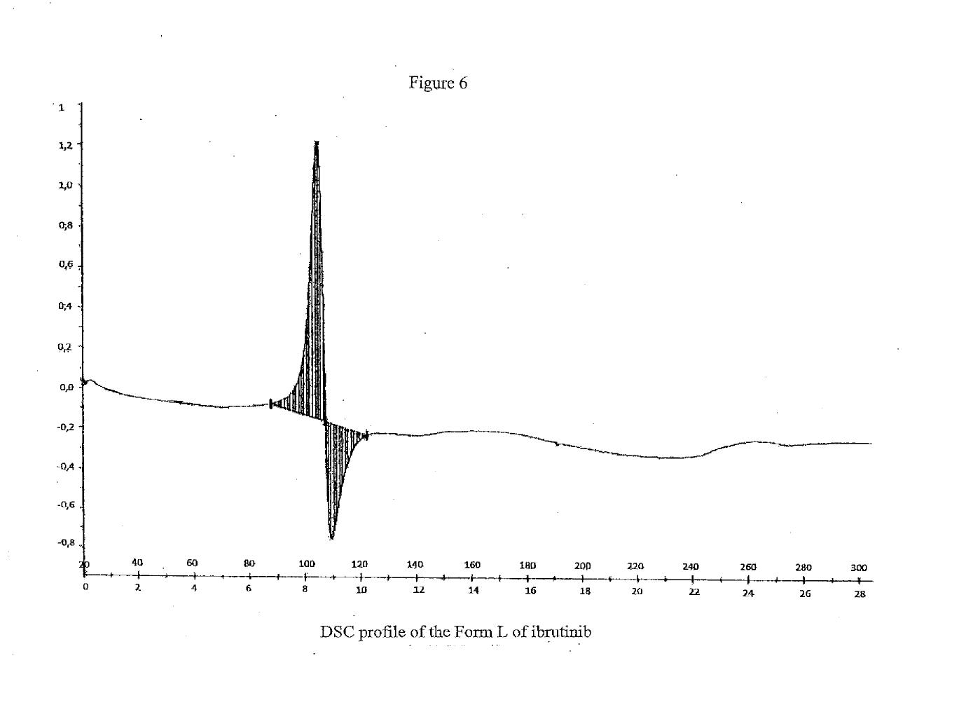

According to a preferred embodiment, the solvate of ibrutinib with 1,2-dimethoxy-ethane is in crystalline form and represents a novel crystalline form of ibrutinib, herein called “Form L”, showing the X-ray diffraction spectrum attached to the present description as FIG. 4, the FT-IR spectrum of FIG. 5 and the DSC profile of FIG. 6.

In particular, the novel Form L of ibrutinib shows the following main peaks:

| Pos. | | | | |
|---|---|---|---|---|
| [°2Th.] | Height [cts] | FWHM [°2Th.] | d-spacing [Å] | Int. Rel. [%] |
| 6.6787 | 2958.98 | 0.1004 | 13.23507 | 90.66 |
| 6.9321 | 1694.32 | 0.1506 | 12.75179 | 51.91 |
| 9.8563 | 235.01 | 0.1338 | 8.97416 | 7.20 |
| 10.3728 | 608.51 | 0.2007 | 8.52841 | 18.64 |
| 10.6814 | 1263.47 | 0.1004 | 8.28269 | 38.71 |
| 10.8263 | 1406.98 | 0.1338 | 8.17220 | 43.11 |
| 12.5982 | 76.74 | 0.2007 | 7.02650 | 2.35 |
| 13.3783 | 1257.52 | 0.1004 | 6.61846 | 38.53 |
| 13.5977 | 1227.74 | 0.1338 | 6.51216 | 37.62 |
| 15.0077 | 101.93 | 0.2342 | 5.90336 | 3.12 |
| 15.5213 | 646.62 | 0.1171 | 5.70915 | 19.81 |
| 15.6574 | 457.84 | 0.0836 | 5.65985 | 14.03 |
| 16.4966 | 151.00 | 0.1338 | 5.37375 | 4.63 |
| 16.8148 | 214.20 | 0.1004 | 5.27277 | 6.56 |
| 17.3130 | 2163.77 | 0.1004 | 5.12217 | 66.30 |
| 17.4179 | 2833.10 | 0.0669 | 5.09153 | 86.80 |
| 17.7776 | 1397.25 | 0.1840 | 4.98932 | 42.81 |
| 18.2364 | 1028.69 | 0.1171 | 4.86481 | 31.52 |
| 18.3932 | 1197.57 | 0.1171 | 4.82370 | 36.69 |
| 18.6861 | 429.93 | 0.1004 | 4.74874 | 13.17 |
| 19.1251 | 472.06 | 0.1338 | 4.64072 | 14.46 |
| 19.4492 | 349.67 | 0.2342 | 4.56413 | 10.71 |
| 20.2046 | 3263.76 | 0.1506 | 4.39516 | 100.00 |
| 20.3189 | 2809.48 | 0.0836 | 4.37069 | 86.08 |
| 20.6131 | 865.75 | 0.1428 | 4.30539 | 26.53 |
| 20.7066 | 743.23 | 0.1020 | 4.29682 | 22.77 |
| 21.5565 | 3045.62 | 0.3060 | 4.11906 | 93.32 |
| 22.1836 | 3193.73 | 0.1632 | 4.00402 | 97.85 |
| 22.3005 | 3097.82 | 0.1428 | 3.98329 | 94.92 |
| 23.1193 | 556.97 | 0.1224 | 3.84404 | 17.07 |
| 23.5168 | 1579.10 | 0.0816 | 3.77996 | 48.38 |
| 23.7528 | 543.90 | 0.1224 | 3.74293 | 16.66 |
| 24.5069 | 62.78 | 0.2448 | 3.62943 | 1.92 |
| 25.4403 | 416.72 | 0.3264 | 3.49834 | 12.77 |
| 26.1500 | 213.55 | 0.2448 | 3.40500 | 6.54 |
| 26.5774 | 386.98 | 0.2040 | 3.35120 | 11.86 |
| 27.0626 | 605.97 | 0.1632 | 3.29221 | 18.57 |
| 27.5144 | 296.96 | 0.1632 | 3.23916 | 9.10 |
| 27.8633 | 353.34 | 0.1428 | 3.19939 | 10.83 |
| 28.3417 | 366.22 | 0.1632 | 3.14646 | 11.22 |
| 28.7403 | 286.08 | 0.2448 | 3.10372 | 8.77 |
| 29.3096 | 60.64 | 0.1632 | 3.04472 | 1.86 |
| 29.8684 | 553.73 | 0.2856 | 2.98902 | 16.97 |
| 30.1973 | 219.49 | 0.1428 | 2.95721 | 6.73 |
| 30.7585 | 124.10 | 0.2448 | 2.90452 | 3.80 |
| 31.3718 | 220.39 | 0.3264 | 2.84913 | 6.75 |
| 31.9224 | 90.00 | 0.2448 | 2.80122 | 2.76 |
| 33.4214 | 196.91 | 0.2448 | 2.67893 | 6.03 |
| 34.1047 | 79.08 | 0.2448 | 2.62681 | 2.42 |
| 35.0086 | 90.13 | 0.4896 | 2.56103 | 2.76 |
| 36.5111 | 142.39 | 0.3264 | 2.45901 | 4.36 |
| 38.3076 | 52.38 | 0.3264 | 2.34772 | 1.60 |
| 39.2997 | 57.36 | 0.3264 | 2.29071 | 1.76 |

The novel Form L contains 1,2-dimethoxy-ethane in the crystal.

The novel Form L of ibrutinib showed to be stable even after mechanical handling, as grinding and kneading, and has a melting point of 104.5° C.

Nevertheless, in some conditions, the Form L converts in Form A or in the amorphous form and for this reason the Form L can be used as intermediate in the preparation of the amorphous form or the Form A.

In fact, it has been observed that by heating the Form L in the presence of humidity, for example by keeping it at 60° C./75% relative humidity, said Form is converting in Form A. The same conversion is obtained by suspending and stirring a suspension of Form L in water for several hours, for example 50-300 hours, preferably about 200-250 hours.

Alternatively, it is possible to obtain the amorphous form of ibrutinib by heating the sample at 60-120 ° C., preferably 80-100° C. for a period of 1-12 hours, preferably 2-10 hours.

The use of the Form L of ibrutinib as an intermediate for the preparation of the amorphous form is a further subject-matter of the invention.

Subject-matter of the invention, according to another aspect thereof, is a process for the preparation of the Form L of ibrutinib, comprising passing isopropyl ether vapors over a saturated solution of ibrutinib in 1,2-dimethoxy-ethane, until obtaining a precipitation and isolating the so-obtained Form L.

The process of the invention can be carried out at room temperature.

The saturated solution can be obtained by dissolving ibrutinib in the solvent at room temperature. The solution is advantageously filtered prior to proceeding to vaporize isopropyl ether and the vaporization time can last 2 to 24 hours, for example about 7-10 hours. The isolation of the Form L can be made by filtration, for example by filtration under vacuum.

In the process described above, any form of ibrutinib can be used as a starting product.

Alternatively, the novel Form L can also be obtained by simply stirring (“slurry”) ibrutinib in 1,2-dimethoxy-ethane. Any form of ibrutinib, can be used. The stirring time ranges from 24 to 100 hours, for example around 50-70 hours. However, the expert in the art is able to follow the progress of the reaction by conventional techniques.

Examples of preparation are provided in the experimental section of the present description.

Form L of ibrutinib, obtainable and/or obtained by the process described above, is a further subject-matter of the invention.

Experimental Section

XRPD

The samples underwent X-ray powder diffraction on the untreated samples.

Instrument: X'Pert PRO

| Scan Axis | Gonio |
|---|---|
| Start Position [°2Th.] | 3.0094 |
| End Position [°2Th.] | 39.9844 |
| Step Size [°2Th.] | 0.0170 |
| Scan Step Time [s] | 12.9218 |
| Scan Type | Continuous |
| PSD Mode | Scanning |
| PSD Length [°2Th.] | 2.12 |
| Offset [°2Th.] | 0.0000 |
| Divergence Slit Type | Fixed |
| Divergence Slit Size [°] | 0.4354 |
| Specimen Length [mm] | 10.00 |
| Measurement Temperature [° C.] | 25.00 |
| Anode Material | Cu |
| K-Alpha1 [Å] | 1.54060 |
| K-Alpha2 [Å] | 1.54443 |
| K-Beta [Å] | 1.39225 |
| K-A2/K-A1 Ratio | 0.50000 |
| Generator Settings | 40 mA, 40 kV |
| Diffractometer Type | 0000000011019590 |
| Diffractometer Number | 0 |
| Goniometer Radius [mm] | 240.00 |
| Dist. Focus-Diverg. Slit [mm] | 100.00 |
| Incident Beam Monochromator | No |
| Spinning | Yes |

FT-IR

The analysis was carried out on non-treated samples by using a Thermo Nicolet 6700 FT-IT spectrometer equipped with Smart performer ZnSe; DTGS Kbr Detector; IR Source; KBr Beam Splitter.

DSC

The analysis was carried out on non-treated samples by using a 200 F3 Maia® DSC

The sample has been weighed in an aluminum container sealed with an aluminum lid. The analysis has been carried out by heating the sample from 25° C. to 350° C. at 10K/minute.

TGA

The analysis was carried out on non-treated samples by using the Mettler Toledo Stare System.

The sample has been weighed in an aluminum container sealed with a perforated aluminum lid. The analysis has been carried out by heating the sample from 25° C. to 450° C. at 10K/minute.

EGA

The analysis has been carried out on gases produced by the TGA.

| Automation | 34 positions of the samples |
|---|---|
| TGA-FTIR | coupled with the Thermo Nicolet 6700 spectrometer |
| “Balance data” | XP5 |
| Measurement range | ≤5 g |
| Resolution | 1.0 μg |
| Weighing accuracy | 0.005% |
| Weighing precision | 0.0025% |

Weights of the internal ring 2

Reproducibility of the control curve: higher than ±10 μg on the whole temperature range

### EXAMPLE 1

General Preparation for the Precipitation Tests

A sample of ibrutinib has been dissolved in 2 ml solvent to obtain a saturated solution, at room temperature or by heating if needed. The suspension was left stirring overnight and then has been filtered on a 0.45 microns Whatman filter. 10 ml anti-solvent has been added to the so-obtained transparent solution at room temperature under mechanical stirring. The precipitate has been isolated by filtration and dried under vacuum.

### EXAMPLE 2

Preparation of the Amorphous Form of Ibrutinib Upon Precipitation

By operating as described in the general procedure of example 1, the amorphous form of ibrutinib is obtained by using 1,2-dimethoxy-ethane as solvent and water as anti-solvent.

### EXAMPLE 3

Preparation of the Amorphous Form of Ibrutinib Upon Precipitation

By operating as described in the general procedure of example 1, the amorphous form of ibrutinib is obtained by using ethanol as solvent and water as anti-solvent.

### EXAMPLE 4

General Preparation for the Evaporation Tests

A sample of 50 mg ibrutinib has been dissolved in 5 ml solvent or a 1/1 (v/v) mixture of two solvents, by heating when needed. The solution has been stirred at room temperature for about 60 minutes, filtered on a 0.45 microns Whatman filter and left evaporating in the following conditions:
Low temperature and room pressure (4-10° C./1 atm)
Room temperature and pressure (17-25° C./1 atm)
High temperature and room pressure (60° C./1 atm)
Room temperature and low pressure (17-25° C./10⁻² atm)
High temperature and low pressure (40° C./10⁻² atm)

### EXAMPLE 5

Preparation of the Amorphous Form of Ibrutinib by Evaporating in Only One Solvent

By operating as described in the general procedure of example 4, in any temperature and pressure condition depicted in example 4, the amorphous form of ibrutinib is obtained by using a solvent selected from 1,4-dioxane and methyl ethyl ketone.

### EXAMPLE 6

Preparation of the Amorphous Form of Ibrutinib by Evaporating in Only One Solvent

EXAMPLE 6.a

By operating as described in the general procedure of example 4, the amorphous form of ibrutinib is obtained by using the temperature and pressure conditions 17-25° C./1 atm in methanol or in acetone.

### EXAMPLE 6.b

By operating as described in the general procedure of example 4, the amorphous form of ibrutinib is obtained by using the temperature and pressure conditions 60° C./1 atm in a solvent selected from 2-butanol, 2-methoxyethanol, acetonitrile, dimethylformamide, dimethylsulfoxide, and ethyl acetate.

### EXAMPLE 6.c

By operating as described in the general procedure of example 4, the amorphous form of ibrutinib is obtained by using the temperature and pressure conditions 17-25° C./10⁻² atm in a solvent selected from 2-butanol, acetonitrile, methylene chloride, methanol, ethanol, nitromethane and ethyl acetate.

### EXAMPLE 6.d

By operating as described in the general procedure of example 4, the amorphous form of ibrutinib is obtained by using the temperature and pressure conditions 40° C./10⁻² atm in a solvent selected from 2-butanol, 2-methoxyethanol, acetonitrile, dimethylformamide, dimethylsulfoxide, ethyl acetate, ethanol and nitromethane.

### EXAMPLE 7

Preparation of the Amorphous Form of Ibrutinib by Evaporation in Mixtures of Solvents

By operating as described in the general procedure of example 4 and using the following mixtures of solvents:
mixture of methyl ethyl ketone/1-2-dimethoxy-ethane
methyl ethyl ketone/1,4-dioxane

the amorphous form of ibrutinib is obtained.

### EXAMPLE 8

The amorphous form of ibrutinib is obtained according to the following examples.

### EXAMPLE 8.a

By operating as described in the general procedure of example 4, at room temperature, at a temperature around 60° C. and room pressure, or at low pressure (about 10−2 atmospheres) at room temperature or about 40° C., in the following mixtures of solvents:
2-propanol/1,4-dioxane;
methyl ethyl ketone/2-propanol;
methyl ethyl ketone/acetonitrile; and
methyl ethyl ketone/ethanol.

### EXAMPLE 8.b

By operating as described in the general procedure of example 4, at a temperature around 60° C. and room pressure, or at low pressure (about 10−2 atmospheres) at room temperature or about 40° C., in the following mixtures of solvents:
methyl ethyl ketone/ethyl acetate;
methyl ethyl ketone/2-butanol;
2-propanol/1,2-dimethoxy-ethane;
2-propanol/ethyl acetate; and
2-propanol/2-butanol.

### EXAMPLE 8.c

By operating as described in the general procedure of example 4, at room temperature or about 40°, in both cases at low pressure (about 10⁻² atmospheres), in the following solvent mixture:
2-propanol/acetonitrile.

### EXAMPLE 8.d

By operating as described in the general procedure of example 4, at a temperature around 60° C. and room pressure or at about 40° C. at low pressure (about 10⁻² atmospheres), in the following solvent mixture:
2-propanol/ethanol. 2-propanol/acetonitrile

### EXAMPLE 8.e

By operating as described in the general procedure of example 4, at room pressure and temperature or at room temperature and low pressure (about 10−2 atmospheres) or at low temperature (4-10° C.), in the following solvent mixture:
methyl ethyl ketone/acetone.

### EXAMPLE 8.f

By operating as described in the general procedure of example 4, at room pressure and temperature or at room temperature and low pressure (about 10−2 atmospheres), in the following solvent mixture:
2-propanol/acetone.

### EXAMPLE 9

Preparation of the Crystalline Form L of Ibrutinib

A sample of ibrutinib has been dissolved in 1,2-dimethoxy-ethane to obtain a saturated solution, at room temperature. The suspension was left stirring overnight and then has been filtered on a 0.45 microns Whatman filter. The so-obtained transparent solution has been exposed to isopropyl ether vapors for 8 days. The precipitate has been isolated by filtration and dried under vacuum, thus providing the Form L of ibrutinib.

### EXAMPLE 10

Preparation of the Crystalline Form L of Ibrutinib

A sample of 1 g ibrutinib has been dissolved in 20 ml 1,2-dimethoxy-ethane to obtain a solution at room temperature. 25 ml isopropyl ether has been added at room temperature, under stirring, to the solution. Thus, the solution has been quickly cooled to 0° C. The precipitate obtained has been isolated by filtration and dried under vacuum and provides the Form L of ibrutinib.

### EXAMPLE 11

Preparation of the Crystalline Form L of Ibrutinib

A sample of 100 mg ibrutinib has been suspended in 1 ml 1,2-dimethoxy-ethane. The suspension was left stirring for 65 hours. The precipitate formed has been isolated by filtration and dried under vacuum, thus providing the Form L of ibrutinib.

### EXAMPLE 12

Stability Tests

The amorphous form, duly dried, demonstrated to be stable over time.

In particular the following tests have been made:
stability at 25° C./40% Relative Humidity for 7 days
stability at 40° C./75% Relative Humidity for 7 days
stability at 25° C./60% Relative Humidity for 7 days
stability at 60° C./40% Relative Humidity for 7 days

In all of the tests, the amorphous form resulted to be stable.

The amorphous form proved to be stable also after grinding and kneading.

1. A process for the preparation of an amorphous form of ibrutinib comprising:
dissolving ibrutinib in a solvent selected from the group consisting of 1,2,-dimethoxy-ethane and ethanol, until obtaining a saturated solution,
adding water to said saturated solution, and
isolating a precipitate from said saturated solution.

2. A process for the preparation of an amorphous form of ibrutinib comprising evaporating a solution of ibrutinib in a solvent selected from the group consisting of 1,4-dioxane, methyl ethyl ketone, methanol, dimethylsulfoxide, ethanol, 2-butanol, acetonitrile, ethyl acetate, nitromethane, 2-methoxyethanol, dimethylformamide and methylene chloride.

3. A process for the preparation of the amorphous form of ibrutinib comprising evaporating a solution of ibrutinib in a solvent mixture selected from the group consisting of methyl ethyl ketone/1,2-dimethoxy-ethane; methyl ethyl ketone/1,4-dioxane; 2-propanol/1,4-dioxane; methyl ethyl ketone/acetone; methyl ethyl ketone/2-propanol; methyl ethyl ketone/ethanol; 2-propanol/acetone; methyl ethyl ketone/ethyl acetate; methyl ethyl ketone/2-butanol; 2-propanol/1,2-dimethoxy-ethane;
2-propanol/ethyl acetate; 2-propanol/2-butanol, 2-propanol/ethanol, and 2-propanol/acetonitrile.

4. The process according to claim 2, wherein said solution is not saturated.

5. The amorphous form of ibrutinib obtainable and/or obtained by the process of claim 1.

6. The amorphous form of ibrutinib obtainable and/or obtained by the process of claim 1, characterized by the FT-IR spectrum of FIG. 2 and by the DSC profile of FIG. 3.

7. (canceled)

8. A pharmaceutical composition comprising the amorphous form of ibrutinib of claim 5 together with conventional carriers and/or excipients.

9. (canceled)

10. (canceled)

11. (canceled)

12. The process according to claim 3, wherein said solution is not saturated.

13. The amorphous form of ibrutinib obtainable and/or obtained by the process of claim 2.

14. The amorphous form of ibrutinib obtainable and/or obtained by the process of claim 3.

15. The amorphous form of ibrutinib obtainable and/or obtained by the process of claim 4.

16. A pharmaceutical composition comprising the amorphous form of ibrutinib of claim 6 together with conventional carriers and/or excipients.

17. A method for treatment and therapy of tumors such as lymphomas and leukemias, comprising administering, to a subject in need thereof, a therapeutically effective amount of the amorphous form of ibrutinib according to claim 5.

18. A method for treatment and therapy of tumors such as lymphomas and leukemias, comprising administering, to a subject in need thereof, a therapeutically effective amount of the amorphous form of ibrutinib according to claim 6.

19. The amorphous form of ibrutinib obtainable and/or obtained by the process of claim 2, characterized by the FT-IR spectrum of FIG. 2 and by the DSC profile of FIG. 3.

20. The amorphous form of ibrutinib obtainable and/or obtained by the process of claim 3, characterized by the FT-IR spectrum of FIG. 2 and by the DSC profile of FIG. 3.
